(19) United States
(12) Patent Application Publication
(10) Pub. No.: US 2004/0006264 A1
(43) Pub. Date: Jan. 8, 2004

(54) Neural prosthetic micro system

(76) Inventors: Mohammad M. Mojarradi, La Canada, CA (US); Erik J. Brandon, Pasadena, CA (US); Jay F. Whitacre, Pasadena, CA (US); Linda Y. Del Castillo, Pasadena, CA (US); Richard A. Andersen, La Canada, CA (US); Travis W. Johnson, Santa Clarita, CA (US); William C. West, S. Pasadena, CA (US)

Correspondence Address: FISH & RICHARDSON, PC, 12390 EL CAMINO REAL, SAN DIEGO, CA, 92130-2081, US

(21) Appl. No.: 10/301,530

(22) Filed: Nov. 20, 2002

Related U.S. Application Data
(60) Provisional application No. 60/349,655, filed on Nov. 20, 2001.
(60) Provisional application No. 60/349,875, filed on Jan. 18, 2002.

(51) Int. Cl. A61N 1/05

(52) U.S. Cl. 600/378; 623/25; 607/48

(57) ABSTRACT

A neural prosthetic micro system includes an electrode array coupled to an integrated circuit (IC) which may include signal conditioning and processing circuitry. The IC may include a high pass filter that passes signals representative of local field potential (LFP) activity in a subject's brain.

### CROSS-REFERENCE TO RELATED APPLICATIONS

This application claims priority to U.S. Provisional Application Serial No. 60/349,655, filed on Nov. 20, 2001, and entitled, “INTEGRATED ELECTRODE ARRAY FOR A NEURO-PROSTHETIC IMPLANT,” and U.S. Provisional Application Serial No. 60/349,875, filed on Jan. 18, 2002, and entitled, “MINIATURIZED BRAIN IMPLANTABLE NEURO PROSTHETIC MICRO SYSTEM.”

### ORIGIN OF INVENTION

The research and development described in this application were supported by NASA under grant number NAS7-1407, DARPA grant number MDA972-00-1-0029, NEI bioengineering grant number 5 R01 EY13337 and ONR grant number N00014-01-0035. The U.S. Government may have certain rights in the claimed inventions.

### BACKGROUND

Limb prostheses may operate in response to muscle contractions performed by the user. Some of these prostheses are purely mechanical systems. Other prostheses may incorporate electronic sensors to measure muscle activity and use the measured signals to operate the prosthesis. These types of prostheses may provide only crude control to users that have control over some remaining limb musculature.

Prosthetic devices and other assistive aids that require control over some remaining limb musculature may not be useful for individuals who have suffered from upper spinal cord injury, extremely debilitating strokes, and neurodegenerative diseases. Prosthetic devices that operate in response to electrical signals measured by a sensor implanted in the subject's brain are being contemplated for assisting these individuals.

### SUMMARY

A micro system for implantation in a subject may include an electrode array bonded to an integrated circuit (IC) including electronic circuitry for conditioning and processing signals obtained by the electrodes. An alignment plate, e.g., a micromachined silicon plate, including holes corresponding to the positions of contact pads on the IC may be bonded to the IC. The electrodes, e.g., wire probes, may be inserted in the holes and bonded to the contact pads. The space between the alignment plate and the IC may be underfilled with a biocompatible material.

Each amplifier in the array may include a filter, e.g., an anti-aliasing filter (AAF), for filtering out a low frequency drift component of signals received from the corresponding electrode. A multiplexer system may multiplex signals sampled from amplifiers in the array and output a single stream of data.

The IC may include a high pass filter that passes relatively low frequency signals, e.g., about 5-100 Hz, which may be representative of local field potential (LFP) activity in the subject's brain. The high pass filter may include a digitally refreshed look-up table (LUT) that stores offset values and gain vectors for each amplifier in the array. The offset values may be converted to analog signals by a digital-to-analog converter (DAC) and presented to the negative terminal of a differential amplifier and subtracted from the signals from the amplifiers provided at the positive terminal of the differential amplifier. The signal output from the differential amplifier may be converted into a digital signal by an analog-to-digital converter (ADC) and processed by a DSP to remove an unwanted low frequency component. The DSP may update the values in the LUT. The portion of the IC including the signal conditioning and processing circuitry may be shielded from corrosive fluids in the subject's brain by plates bonded to the IC and/or a polymer coating.

The penetration depth of the electrodes in the subject's brain may be controlled by an adjustable plate. An electrode plate including machined holes having the same pitch as electrodes in the electrode array may be mounted on the micro system such that the electrodes can travel through the holes. Actuators connected between the electrode plate and the IC substrate may be used to control the position of the electrode plate, and thereby the effective length of the electrodes. The actuators may be microbatteries with solid state electrolytes. The microbatteries may expand or contract depending on the charge stored in the battery. Microbatteries may be stacked to increase the potential range of motion between the electrode plate and the IC. A back plate may be provided on the side of the IC opposite the electrodes. Actuators connected between the back plate and the IC. The back plate may push against a surface in the subject opposite the tissue in which the electrodes are implanted, thereby pushing the electrodes deeper into the tissue. A servo control section may be included in the IC which provides signals to the actuators in response to the signal strength of signals received from the electrodes.

### BRIEF DESCRIPTION OF THE DRAWINGS

FIG. 1 is a side view of a neural prosthetic micro system.

FIG. 2 is a block diagram of signal processing circuitry in an mixed signal integrated circuit (IC) in the micro system.

FIG. 3 is a perspective view of a partially fabricated sensor including an alignment plate and electrode array.

FIG. 4 is a schematic diagram of an amplifier in the IC.

FIG. 5 is an exploded perspective view of a spiral micro-coil antenna.

FIG. 6 is a perspective view of a micro system including electromechanical actuators for controlling penetration depth of electrodes in a subject's brain.

FIGS. 7A-7F show a process flow for fabrication of microbatteries which may be used as actuators.

FIG. 8 is a sectional view of a subcutaneously implanted IC connected to recording electrodes implanted in a subject's brain.

### DETAILED DESCRIPTION

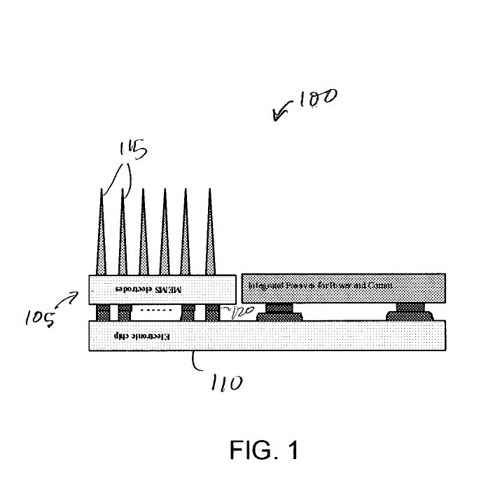

FIG. 1 illustrates a neural prosthetic micro system including an electrode array integrated with an integrated circuit (IC). The system may be implanted in a subject's brain. Alternatively the IC system can be implanted subcutaneously with a connector to recording electrodes implanted in the brain. The electrodes in the array may pick up signals from neurons in the cerebral cortex of the brain.

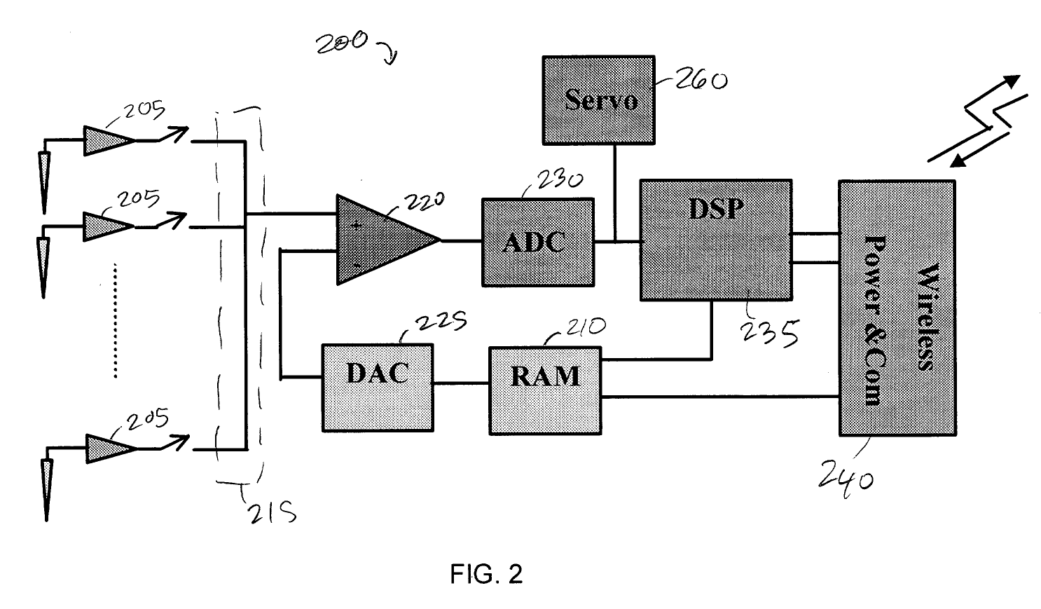

As shown in FIG. 2, the IC 110 may include electronic circuitry 200 for processing the signals obtained by the electrodes. The extracted and processed signals may then be further processed and/or analyzed by an external system and used to control a prosthetic device based on the subject's intention as recorded by the neural signals. The processed signals can be used for a variety of applications, among them, controlling a robotic limb, a computer for communication, electrical stimulators surgically implanted in the patients' limbs to allow movement of their own limbs, or an autonomous vehicle.

The electrode array may be a Micro Electro Mechanical Systems (MEMS)-based sensor. A MEMS system may be fabricated using IC processing technologies. Electrodes in the MEMS sensor 105 may be constructed from a semiconductor material, e.g., silicon, and coated with platinum at the tips and contact pads and a silicon nitride insulator along the shank of the electrode. The electrode array may include one hundred electrodes in a 10×10 array. The electrodes may be about 1.5 mm long and be separated by a spacing of about 400 microns. Bionic Technologies, LLC of Salt Lake City, Utah produces electrode arrays of this type. Alternatively the electrodes may be a bundle of microwires inserted into the brain. These microwires may be inserted using stereotaxic guided surgeries similar to those used currently in deep brain stimulation neurosurgeries.

The IC 110 may include one hundred analog amplifier channels 205 (one amplifier per electrode) to interface with the electrodes 115. The IC may include a micro-pad array including contact pads 120 arranged in a 10×10 matrix having the same pitch as the electrodes. The micro-pad array may enable direct connection between individual electrodes 115 and analog amplifiers 205 in the IC 110. Alternatively, the micro-pad array may lead to a connector which connects the array to the IC device.

The MEMS sensor may be electrically and mechanically bonded to the IC using a flip chip bonding technique. The IC may include an array of contact bumps on every pad of the micro-pad array. The flip chip connection may be formed using solder or a conductive adhesive.

A solder bumped IC may be attached to the MEMS sensor by a solder reflow process. After the IC is soldered, underfill may be added between the IC and the MEMS sensor. The underfill may be a biocompatible epoxy that fills the area between the die and the carrier, surrounding the solder bumps and isolating them from corrosive fluids in the subject's brain. The underfill may control stress in the solder joints caused by the difference in thermal expansion between the IC and the MEMS sensor. Once cured, the underfill may absorb the stress, reducing the strain on the solder bumps, which may increase the life of the finished package. The spacing between the MEMS sensor and the chip may be sealed with a final coat of parylene.

In an alternative implementation, electrodes may be connected individually to the micro-pads in the array. The electrodes, e.g., wire probes, may be inserted into the solder bumps through a reflow process in which the probes are fixed in place and electrically connected to pads in the micro-pad array. The physical mounting and electrical connection is provided by the solder bumps (which can be made lead-free, to be biocompatible). Encapsulation and underfill material can also be used for further protection.

A wide variety of probe tip materials may be used. A biocompatible metal or alloy that can be drawn into a fine wire, e.g., tungsten, may be used as the probe material. The wire probe may be drawn to a desired length. This flexibility in the selection of material and length may provide the capability of tailoring the probe and optimizing its impedance characteristics and ability to pick up signals around the neurons.

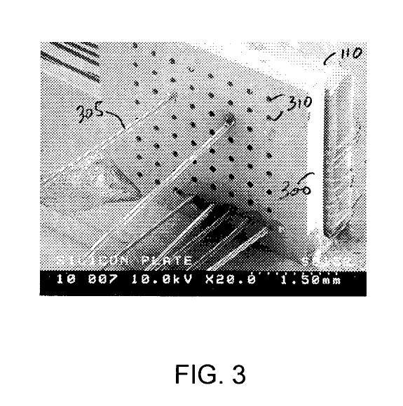

A silicon plate 300 may be used to align and support the individually inserted wire probes 305, as shown in FIG. 3. The silicon plate 300 may be fabricated from a silicon wafer, e.g., about 550 microns thick. Holes 310 corresponding in position to the contact bumps may be micromachined into the silicon plate using MEMS fabrication techniques. A conductive epoxy may be placed over the conductive bumps. The plate may be separated from the IC with glass beads. The plate may be aligned with the IC and attached using a flip chip bonding technique. The wire probes may then be inserted into the holes and the conductive epoxy to provide an electrical contact between the wire probes and corresponding conductive bumps in the array. The space between the silicon plate and the IC may be sealed with biocompatible epoxy and a final coat of parylene.

The electrodes may be used to record spike trains from individual neurons (single units or “SUs”). Spike trains may be used to predict a subject's intended movements, e.g., a reach or saccade. Spike trains may be relatively high frequency events, e.g., several kHz. The amplifiers in the IC may be followed by a corresponding array of high pass filters to pass the relatively high frequency SU activity and attenuate lower frequency activity. The high pass filters may be single pole integrated filters, which include a combination of resistors and capacitors. The high pass filters may have a relatively low cutoff of about 100 Hz, which may be realized through the use of relatively high resistor and capacitor values.

The electrodes may also be used to record local field potential (LFP) activity. LFP is an extracellular measurement that represents the aggregate activity of a population of neurons, which may also encode a subject's intended movements. The LFP measured at an implanted electrode during the preparation and execution of a task has been found to have a temporal structure that is approximately localized in time and space.

Temporal structure is a general term that describes patterns in activity over time. Temporal structure localized in both time and frequency involves events that repeat approximately with a period, T, during a time interval, after which the period may change. For example, the period may get larger, in which case the frequency could get smaller. However, for the temporal structure to remain localized in frequency as it changes in time, large changes in the frequency of events cannot occur over short intervals in time.

Information provided by the temporal structure of the LFP of neural activity appears to correlate to that provided by SU activity, and hence may be used to predict a subject's intentions. Unlike SU activity, measuring LFP activity does not require isolating the activity of a single unit. Accordingly, it may be advantageous to use LFP activity instead of, or in conjunction with SU activity to predict a subject's intended movement in real time.

Unlike spikes, LFP activity occurs at relatively low frequencies, e.g., in a range of approximately 5 Hz to 200 Hz. The micro system 100 may be used to record LFP activity in this relatively low frequency range, e.g., under about 100 Hz. These low frequencies render the traditional analog high pass filters, outlined above, impractical because of the requirement of very large values of the resistive components. There may be a significant mismatch between the component values and the corresponding noise associated with the values, which may significantly reduce the signal to noise ratio (SNR) of the system.

In the embodiment shown in FIG. 2, the IC 110 may include a system which performs the low frequency cutoff high pass filter function without the array of high pass filters. The system may digitally measure low frequency offset voltages of the brain signals obtained by the electrodes and periodically store the offset values in a memory bank including a look up table (LUT) 210. The data stored in the LUT may be used to produce an error vector that is subtracted from the actual value of the signal from the brain in real time. Since the value of the low frequency offset may change as a function of time, the subtraction of this offset from the original signal performs the equivalent function of a low cut off frequency high pass filter.

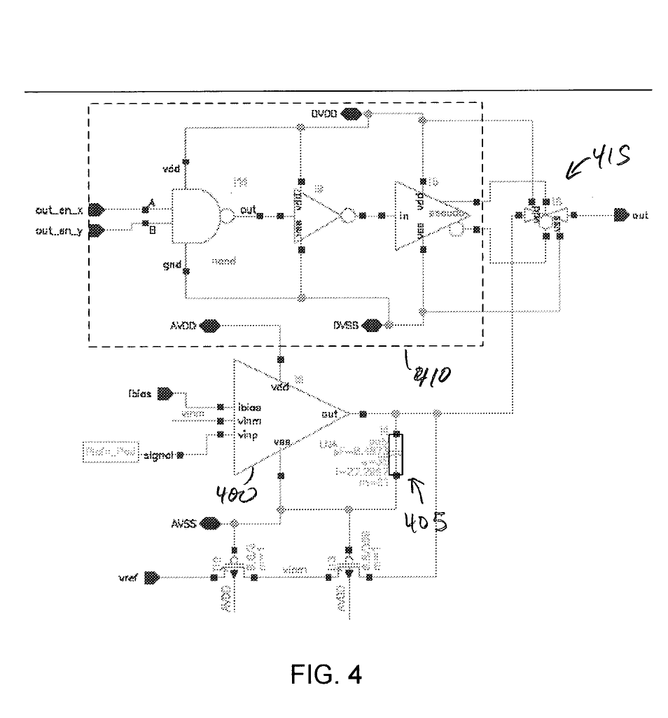

The amplifiers 205 in the array may include analog amplifiers 400 with a limited gain, e.g., of approximately 50 V/V, as shown in FIG. 4. Each amplifier 205 may include a low pass anti-aliasing filter (AAF) 405. The AAF may have a cutoff frequency of approximately 10 kHz.

The amplifier channels may be selected using a digital select circuit 410 and a multiplexer switch 415. The output of each amplifier channel may be connected to a multiplexing system 215. The output of the multiplexing system 215 may be a single channel of sampled time domain multiplexed data. The AAFs may prevent a shadowing effect caused by frequencies that are a step multiple of the clock frequency used to multiplex the signals. The AAFs may act as low pass filters that suppress such high frequencies, e.g., frequencies higher than about 10 kHz.

The data from the multiplexing system 215 may be channeled to a positive terminal of a differential amplifier 220. The negative terminal of the differential amplifier may be connected to the LUT 210 including a look-up table (LUT) through a digital-to-analog converter (DAC) 225.

The LUT 210 may store offset values for each amplifier in the amplifier array. The DAC 225 may present this information as an analog signal to the differential amplifier 220, which may use this signal to subtract unwanted low frequency drift of the signals from the sensor and perform a low cutoff frequency high pass filtering function.

The LUT 210 may also store gain vectors for each amplifier in the array. The gain vectors may be presented to the differential amplifier as the corresponding signal from a amplifier channel is passing through the differential amplifier. The differential amplifier may have a variable gain controlled by these gain vectors. Controlling the gain of the differential amplifier in this manner may prevent the saturation of the differential amplifier and optimize the signal strength from every amplifier channel.

The signal from the differential amplifier 220 may be passed through an analog-to-digital converter (ADC) 230 and processed by a Digital Signal Processing (DSP) unit 235. The DSP may digitally extract the unwanted low frequency portion of the signal from each channel and assign a gain vector to each of the pre amplifiers. The DSP may also perform spike sorting and data compression and prepare data for transmission. The DSP may also perform digital filtering operations to separate out the LFP data and the spike data from the broad band signal from the differential amplifier. The data from the DSP may then be passed off of the chip to an external system for further processing and analysis.

Initial values for gain and offset for each of the amplifier channels may be determined empirically during system calibration and stored in the LUT 220. The DSP 235 may digitally refresh the LUT with the digitally extracted low frequency offset values and assigned gain vectors obtained during operation. The cut off frequency is directly proportional to the update rate of the look-up table and can be digitally controlled by the system to very low frequencies. Also, this cutoff frequency may be the same for all elements of the array, eliminating any mismatch due to physical components.

The combination of the differential amplifier, ADC, DSP, LUT and DAC may produce a servo track that constantly monitors the offset and gain uniformity of each channel.

The IC 110 may include a power and communication section 240 that can receive power and transfer signals wirelessly. The power and communication section 240 may include a dipole antenna, e.g., a bond wire. The length of such an antenna is a function of the frequency. The wire may be coated with parylene to provide insulation.

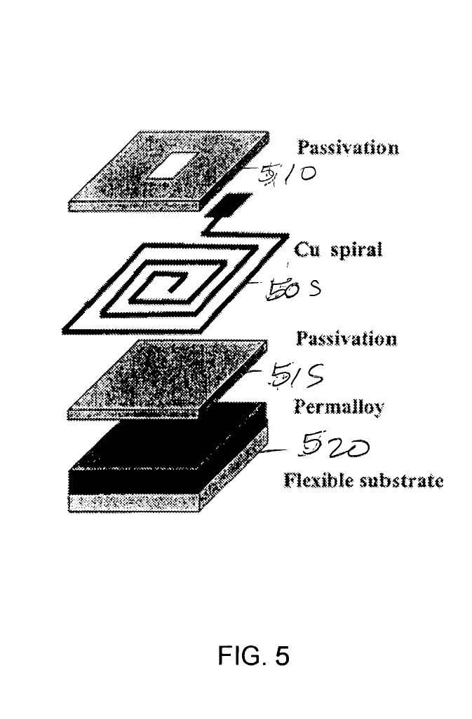

In an alternative implementation, a spiral micro-coil 505, such as that shown in FIG. 5, may be used to transfer signals and power. The micro-coil 505 may be sandwiched between passivation layers 510 and 515 and printed on a flexible substrate 520. The micro-coil may be attached to the chip during the assembly process using two bond wires. A small removable plastic tape may be used to attach the micro-coil to the assembly prior to physical insertion in the brain. The tape may be removed after the micro system is physically placed in the brain. The micro-coil may then be placed under the membrane surrounding the brain.

Another coil may be placed on the exterior surface of the skull. The external coil antenna may be connected to a utility pack that contains the electronics for transmitting power to the nested micro-coil. The interaction between the two coils may be similar to that of a transformer, except that the coefficient of coupling may be relatively low (e.g., 0.1 instead of 1) due to the gap between the primary and secondary windings.

The electrodes may be inserted through the outer layer of the brain, which includes the dura and arachnoid layers, or, alternatively, these layers may be surgically removed prior to implantation.

The proximity of the electrode tips to target neurons may significantly affect the sensitivity of the sensor. Determining and achieving an optimal penetration depth may be difficult at the time of implantation.

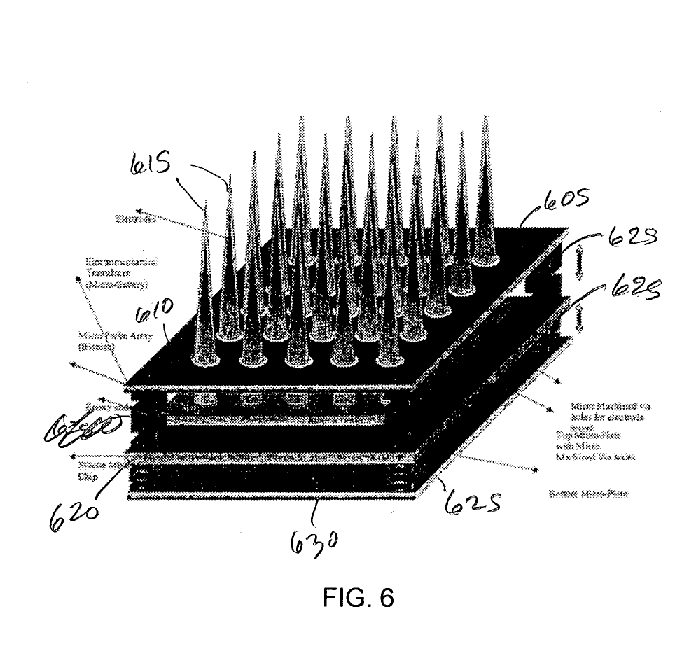

A mechanically adjustable, micro-machined plate 605, such as that shown in FIG. 6, may be used to control the penetration depth of electrodes in the brain. The electrode plate may include machined holes 610 having the same pitch as electrodes 615 in the electrode array. The electrode plate may be mounted on the micro system such that the electrodes can travel through the holes. The electrode plate may be positioned between the IC 620 and the brain. The relative distance between the IC and the electrode plate can be adjusted with the aid of electro-mechanical actuators 625, which may be connected between the electrode plate and the IC at four corners. The actuators take electrical signals from the IC and translate them into mechanical displacement for the electrode plate. The effective penetration depth of the electrodes in the brain can be controlled by moving the electrode plate in relation to the IC.

The micro system may be implanted in many different regions of the brain. In an implementation, the micro system may be implanted in a sulcus, which is a fold in the cortex. Another plate 630 may be placed on the back of the micro system, opposite the electrodes, and used to control the inward motion of the electrodes. Another set of actuators may be connected between the back plate and the IC. The back plate may push against a surface of the sulcus opposite the electrodes and force the electrodes further into the brain matter.

An electronic servo system 260 may be included in the IC (FIG. 2). The servo system may assess the neural signal strength from the electrodes and use this information to readjust the electrode depth to enhance the signal strength.

The actuators 630 may be lithium (Li) microbatteries including a solid state electrolyte. Li microbatteries may expand in thickness as they are charged. A Li microbattery may be designed to expand up to 50% of its uncharged thickness. Other kinds of batteries, like Ni-Hydrogen, may produce a even larger expansion coefficient. A series of micro-batteries may be stacked on top of each other to achieve larger motion.

Compared to other electromechanical actuators, a microbattery actuator may require a relatively low voltage (e.g., about 3V) to expand. Also, a solid state microbattery may retain its shape for as long as it stays charged.

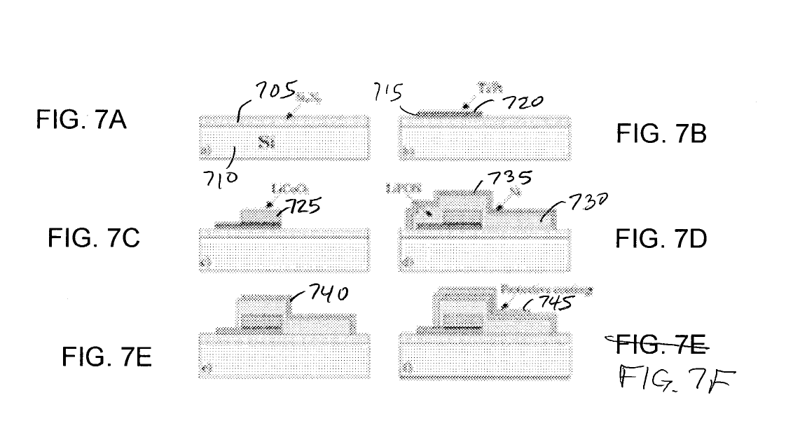

FIGS. 7A-7F shows a process flow for an exemplary Li microbattery. For this microbattery, a 2 μm low-stress silicon nitride film 705 was deposited on Si <100> substrates 710, as shown in FIG. 7, by chemical vapor deposition to provide electrical isolation between the microbattery cells. The substrates were then patterned with negative photoresist to define the cathode current collectors. On the patterned photoresist, a 10 nm Ti adhesion film 715 was deposited on the substrate, followed by the deposition of a 200 nm Pt film 720, as shown in FIG. 7B. The wafers were immersed in acetone or photoresist stripper to remove the photoresist and lift off the excess Ti/Pt film, thereby defining the cathode current collectors. In some cases, the lift-off was facilitated by briefly immersing the samples in a sonicated acetone bath.

To define the microbattery cathodes, the substrates were again patterned with negative resist, yielding square openings in the photoresist 50-100 μm on a side over the cathode current collectors. A film of LiCoO₂ 725 was sputtered over the photoresist, and the wafers were immersed in acetone to remove the photoresist and lift off the excess LiCoO₂, as shown in FIG. 7C. The LiCoO₂ films were moisture sensitive, so the lift-off procedure was performed in a dry room to prevent moisture condensation in the acetone from contaminating the films. Photoresist stripper could not be used since it reacted with the LiCoO₂ film as well. In some cases, following patterning of the cathode features, the substrates were heated to 300° C. for one hour to decrease lattice strain and increase grain size of the nanocrystalline as-sputtered LiCoO₂ films. Whereas the ORNL process requires 700° C. anneal to yield high capacity cathode performance, the 300° C. anneal used here is much more amendable to back-end Si processing, at the cost of lower rate capability of the cathode film.

A Li_{3.3}PO_{3.8}N_{0.22} solid electrolyte film 730 (prepared by RF magnetron sputtering Li₃PO₄ in N₂), was then deposited over the substrates to a thickness of 500-2000 nm, as shown in FIG. 7D. Without breaking vacuum, a 150 nm Ni blocking anode film 735 was subsequently deposited on the solid electrolyte film to protect it from reaction with ambient moisture during removal from the sputter chamber and further photolithography steps. The Ni film was patterned with positive photoresist. The Ni film was then ion milled in Ar for 20 minutes at 750 V and 150 mA to define the Ni anode current collectors and contact pads, shown in FIG. 7D.

To open vias in the solid electrolyte over the cathode contact pads, the wafers were patterned with negative resist so that the only unexposed areas on the samples were over the cathode contact pads. When the photoresist was developed, the uncrosslinked resist dissolved leaving the solid electrolyte exposed to the developer solution, which aggressively attacked the solid electrolyte film. The resist was removed with acetone, yielding the unpassivated full cell microbatteries. Alternatively, after the deposition of the electrolyte film, the wafer can be removed from the sputter chamber and patterned and etched to open vias to the cathode current collector. Deposition and patterning of the Ni film is then performed as usual. Using this method, adjacent cells can be patterned in series for multicell batteries.

In some cases, an encapsulation film was incorporated into the cell design, as shown in FIG. 7E. Presently the encapsulation film employed is a 1 μm sputtered film of Lipon, though a parylene deposition and a patterning process are currently under development in these laboratories.

The micro system may be exposed to corrosive fluids while in the brain. The passivation fill between the MEMS sensor and the electronics under it may protect the electronics from corrosion. The portion of the electronics section of the IC not under the MEMS sensor may be shielded against corrosion with plates micro-machined to the same shape as the area of the exposed electronics. The plates may be attached to the exposed areas of the IC to cover and shield the exposed electronics.

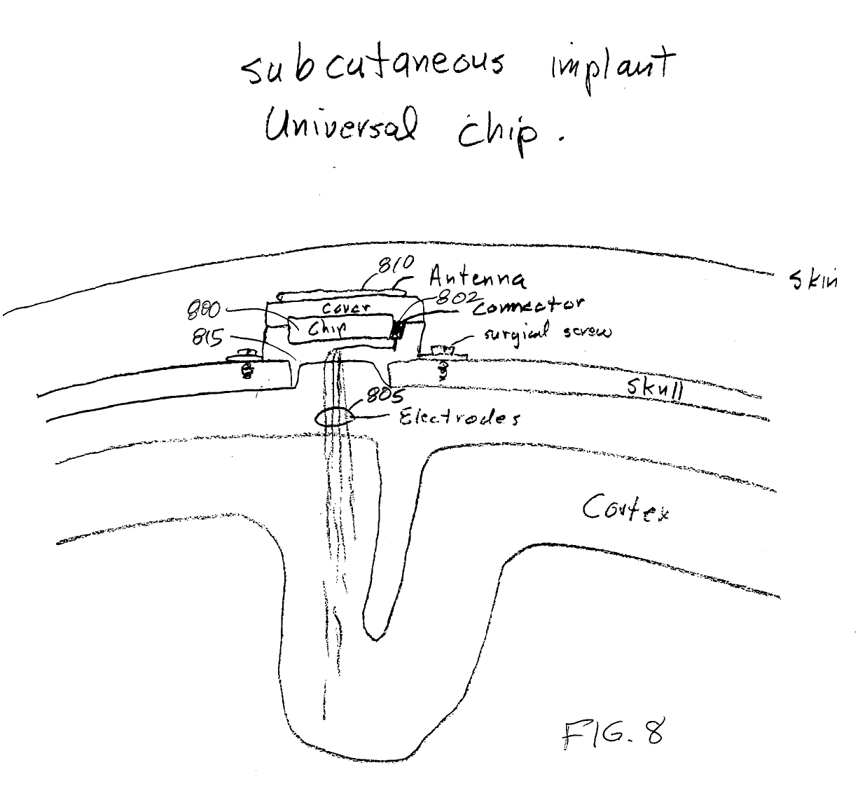

In an alternative implementation, the IC 800 may be implanted sub-cutaneously and, through a connector 802, could be used with a variety of implanted recording electrodes 805 and/or electrode arrays, as shown in FIG. 8. The IC 800 may include or be connected to an antenna 810 for telemetry. The antenna may also be implanted subcutaneously. The advantage of a subcutaneous implant is the reduced potential brain damage from the insertion of a large chip into the brain. Also the heat generated by the system may not interfere with brain function, being located between the scalp and the skin.

The electrodes can also be introduced into the brain by less invasive methods than implanting a chip in the brain. For example, a small burr hole 815 can be made in the skull, a guide tube needle can be inserted through the hole and through the underlying dura, and microelectrode recording wires can be advanced into the brain.

Stereotaxic placement of the wires can be achieved using co-registration of MRIs, CT scans and the coordinates on a stereotaxic frame. This technique is commonly used in brain surgeries (for instance for placement of deep brain stimulators for Parkinson's disease). They are performed with the patient awake, with local anesthesia at the incision and pressure points where the stereotaxic frame contacts the patient's skull.

Moreover, recordings can be made during insertion of the electrodes and the patient can be asked to try to think about movements. This approach can be used to optimize functionally the placement of the electrodes. The less invasive nature of the stereotaxic surgery allows for the patients to remain conscious, since the surgery is less invasive than, for instance, implanting the entire system in the brain. This latter procedure would likely require a craniotomy of larger diameter and dural resection under general anesthesia.

The IC may be mounted in a housing which may be placed near, or over, the location of the burr hole for the electrode implant. The electrodes would be connected to the chip system with a connector. Alternatively, a device may be placed between the chip and wires that would allow for the movement of the wires for fine tuning their locations in the brain after surgery. This fine adjustment could be made on a regular basis, and could be realized by a number of techniques including the microbattery actuators described above.

The IC 800 be incased in ceramic, paralene, glass, metal, or other biocompatible materials. The antenna 810 may be part of the IC 800, or may be a wire with paralene coating implanted subdurally between the skull and overlying skin, and attached to the IC 800, directly or via a connector.

The DSP 235, or alternatively, another DSP in the IC, may be used to further process the filtered signals. The measured waveform(s), which may include frequencies in a range having a lower threshold of about 1 Hz and an upper threshold of from 5 kHz to 20 kHz may be digitally filtered into different frequency ranges. For example, the waveform may be filtered into a low frequency range of say 1-20 Hz, a mid frequency range of say 15-200 Hz, which includes the beta (15-25 Hz) and gamma (25-90 Hz) frequency bands, and a high frequency range of about 200 Hz to 1 kHz, which may include unsorted spike activity. The DSP may perform a spike sorting operation on data in this range.

The digitized LFP and spike (SU) signals may be represented as spectrograms. The spectrograms may be estimated by estimating the spectrum for the data in a time window, translating the window a certain distance in time, and repeating. Although SU activity is a point process composed of discrete events in time (action potentials) in contrast to continuous processes such as the LFP that consist of continuous voltage changes, both may be analyzed using similar methods.

The DSP may estimate the spectral structure of the digitized LFP and spike signals using multitaper methods. Multitaper methods for spectral analysis provide minimum bias and variance estimates of spectral quantities, such as power spectrum, which is important when the time interval under consideration is short.

With multitaper methods, several uncorrelated estimates of the spectrum (or cross-spectrum) may be obtained from the same section of data by multiplying the data by each member of a set of orthogonal tapers. A variety of tapers may be used. Such tapers include, for example, Parzen, Hamming, Hanning, Cosine, etc.

In an embodiment, the Slepian functions are used. The Slepian functions are a family of orthogonal tapers given by the prolate spheroidal functions. These functions are parameterized by their length in time, T, and their bandwidth in frequency, W. For choice of T and W, up to K=2TW-1 tapers are concentrated in frequency and are suitable for use in spectral estimation.

For an ordinary time series, xₜ, t=1, . . . , N. The basic quantity for further analysis is the windowed Fourier transform

$${\tilde{x}}_{k}^{( X )} ( f ) : {\tilde{x}}_{k}^{( X )} ( f ) = {\sum }_{1}^{N} {w}_{t} ( k ) {x}_{t} exp ( - 2 π ⅈ f t )$$

where wₜ(k) (k=1, 2, . . . , K) are K orthogonal taper functions. For the point process, consider a sequence of event times {τⱼ}, j=1, . . . , N in the interval [0,T]. The quantity for further analysis of point processes is also the windowed Fourier transform, denoted by

$${\tilde{x}}_{k}^{( N )} ( f ) : {\tilde{x}}_{k}^{( N )} ( f ) = {\sum }_{j = 1}^{N} {w}_{{τ}_{j}} ( k ) exp ( - 2 π ⅈ f {τ}_{j} ) - \frac{N ( T )}{T} {\tilde{w}}_{0} ( k )$$

where w₀(k) is the Fourier transform of the data taper at zero frequency and N(T) is the total number of spikes in the interval.

When averaging over trials we introduce an additional index, I, denoting trail number \tilde{x}_{k,i}(f).

When dealing with either point or continuous process, the multitaper estimates for the spectrum Sₓ(f), cross-spectrum S_{yx}(f), and coherency C_{yx}(f) may be given by:

$$\begin{matrix}\begin{matrix}{S}_{x} ( f ) = \frac{1}{K} {\sum }_{k = 1}^{K} {\vert {\tilde{X}}_{k} ( f ) \vert}^{2} \\ {S}_{yx} ( f ) = \frac{1}{K} {\sum }_{k = 1}^{K} \vert \tilde{y} k ( f ) {\tilde{x}}_{k}^{*} ( f ) \vert\end{matrix} \\ {C}_{y x} ( f ) = \frac{{S}_{yx} ( f )}{\sqrt{{S}_{x} ( f ) {S}_{y} ( f )}}\end{matrix}$$

The auto- and cross-correlation functions may be obtained by Fourier transforming the spectrum and cross-spectrum. In an alternate embodiment the temporal structure of the LFP and SU spectral structures may be characterized using other spectral analysis methods. For example, filters may be combined into a filter bank to capture temporal structures localized in different frequencies. As an alternative to the Fourier transform, a wavelet transform may be used to convert the date from the time domain into the wavelet domain. Different wavelets, corresponding to different tapers, may be used for the spectral estimation. As an alternative to calculating the spectrum on a moving time window, nonstationary time-frequency methods may be used to estimate the energy of the signal for different frequencies at different times in one operation. Also, nonlinear techniques such as artificial neural networks (ANN) techniques may be used to learn a solution for the spectral estimation.

The DSP may generate a feature vector train, for example, a time series of spectra of LFP, from the input signals. The feature vector train may be transmitted to a decoder and operated on to predict the subject's intended movement, and from this information generate a high level control signal.

A number of embodiments have been described. Nevertheless, it will be understood that various modifications may be made without departing from the spirit and scope of the invention. Accordingly, other embodiments are within the scope of the following claims.

1. An apparatus adapted to be implanted in a subject, the apparatus comprising:
a chip including a plurality of amplifiers arranged in an array; and
a plurality of electrodes, each electrode coupled to a corresponding one of the amplifiers.

2. The apparatus of claim 1, wherein each amplifier includes a filter operative to filter out a low frequency drift component from a signal received from the electrode coupled to said amplifier.

3. The apparatus of claim 2, wherein said low frequency drift component comprises a frequency in a range of from about 1 Hz to about 3 Hz.

4. The apparatus of claim 2, wherein said filters comprise anti-aliasing filters.

5. The apparatus of claim 1, further comprising a high pass filter.

6. The apparatus of claim 5, wherein the high pass filter is operative to pass signals having a frequency below about 200 Hz.

7. The apparatus of claim 6, wherein the high pass filter is operative to pass signals having a frequency greater than about 5 Hz.

8. The apparatus of claim 5, wherein the high pass filter is operative to pass signals representative of local field potential (LFP) activity.

9. The apparatus of claim 5, wherein the high pass filter comprises a look-up table including an offset value for each amplifier in the array.

10. The apparatus of claim 9, wherein the look-up table comprises a gain vector for each amplifier in the array.

11. The apparatus of claim 9, further comprising a digital signal processor (DSP) operative to update values in the look-up table.

12. The apparatus of claim 1, further comprising a multiplexer system coupled to each amplifier in the array and operative to output a stream of data comprising signals sampled from amplifiers in the array.

13. The apparatus of claim 12, further comprising a digital-to-analog converter (DAC) coupled to an output of the look-up table and operative to convert an offset value from the look-up table into an analog signal.

14. The apparatus of claim 13, further comprising a differential amplifier including:
a first input terminal coupled to an output of the multiplexer system;
a second input terminal coupled to an output of the DAC; and
an output terminal.

15. The apparatus of claim 14, further comprising:
an analog-to-digital converter (ADC) coupled to the output terminal of the differential amplifier; and
a digital signal processor (DSP) coupled to an output of the ADC.

16. The apparatus of claim 15, wherein the DSP is operative to extract an unwanted low frequency portion of signals from the amplifiers.

17. The apparatus of claim 16, wherein the DSP is further operative to sort signals representative of spike activity.

18. The apparatus of claim 1, wherein the chip comprises an integrated circuit (IC) including signal processing circuitry.

19. The apparatus of claim 18, further comprising a shield attached to the chip over the signal processing circuitry, said layer being operative to shield said circuitry from fluids in the subject.

20. The apparatus of claim 19, wherein the shield comprises a plate.

21. The apparatus of claim 19, wherein the shield comprises a polymer coating.

22. An apparatus adapted to be implanted in a subject, the apparatus comprising:
a plurality of electrodes;
a substrate;
a plate including a plurality of holes, wherein a plurality of said electrodes extend through corresponding holes in the plate; and
an actuator between the substrate and the plate, the actuator operative to expand in response to receiving a signal, thereby decreasing an effective length of the electrodes extending through the holes.

23. The apparatus of claim 22, wherein the actuator comprises a microbattery including a solid state electrolyte.

24. The apparatus of claim 22, wherein the actuator comprises a plurality of stacked microbatteries, wherein said microbatteries include a solid state electrolyte.

25. The apparatus of claim 22, further comprising a plurality of actuators connected between the substrate and the plate at different locations.

26. The apparatus of claim 22, wherein the substrate comprises an integrated circuit (IC) including a servo control section coupled to the electrodes and the actuators, wherein the servo control section is operative to provide signals to the actuator in response to a signal strength of signals received from the electrodes.

27. An apparatus adapted to be implanted in a subject, the apparatus comprising:
a substrate having a first side and a second side, the second side being opposite the first side;
a plurality of electrodes positioned adjacent to the first side of the substrate;
a plate positioned adjacent to the second side of the substrate; and
an actuator between the substrate and the plate, the actuator operative to expand in response to receiving a signal.

28. The apparatus of claim 27, wherein the actuator comprises a microbattery including a solid state electrolyte.

29. The apparatus of claim 27, wherein the actuator comprises a plurality of stacked microbatteries, wherein said microbatteries include a solid state electrolyte.

30. The apparatus of claim 27, further comprising a plurality of actuators connected between the substrate and the plate at different locations.

31. The apparatus of claim 27, wherein the substrate comprises an integrated circuit (IC) including a servo control section coupled to the electrodes and the actuators, wherein the servo control section is operative to provide signals to the actuator in response to a signal strength of signals received from the electrodes.

32. A method for fabricating an implant, the method comprising:
coupling a contact bump to each of a plurality of amplifiers in an integrated circuit (IC) on a substrate;
bonding an alignment plate to the substrate, the alignment plate including a plurality of holes corresponding in position to the plurality of contact bumps;
inserting a plurality of wire probes into corresponding holes in the alignment plate; and
bonding each wire probe to a corresponding contact bump.

33. The method of claim 32, wherein said bonding the alignment plate comprises depositing a conductive epoxy on each contact bump.

34. The method of claim 32, further comprising underfilling a space between the alignment plate and the substrate with a biocompatible material.

35. The method of claim 32, wherein the alignment plate comprises a micromachined silicon plate.

36. A method comprising:
implanting a device including a plurality of electrodes into a subject during an implantation operation; and
changing a penetration depth of electrodes implanted in the subject after the implantation operation.

37. The method of claim 36, wherein said changing comprises changing an effective length of the electrodes.

38. The method of claim 37, wherein said changing the effective length of the electrodes comprises expanding one or more actuators positioned between a substrate and an electrode plate including a plurality of holes through which the electrodes extend.

39. The method of claim 38, wherein said expanding comprises increasing a voltage stored in a microbattery including a solid state electrolyte.

40. The method of claim 36, wherein said changing comprises pushing against a surface opposite the electrodes.

41. The method of claim 40, wherein said pushing comprises expanding actuators between a substrate having a first side adjacent the electrodes and a plate adjacent a side of the substrate opposite the first side.

42. The method of claim 41, wherein said expanding comprises increasing a voltage stored in a microbattery including a solid state electrolyte.

43. A micro system adapted to be implanted in a subject, the micro system comprising:
a chip including a plurality of amplifiers arranged in an array;
a plurality of electrodes, each electrode coupled to a corresponding one of the amplifiers; and
a high pass filter operative to pass signals representative of local field potential (LFP) activity.

44. The micro system of claim 43, wherein the high pass filter comprises a look-up table including an offset value for each amplifier in the array.

45. The micro system of claim 44, further comprising:
a multiplexer system coupled to each amplifier in the array and operative to output a stream of data comprising signals sampled from amplifiers in the array;
a digital-to-analog converter (DAC) coupled to an output of the look-up table and operative to convert an offset value from the look-up table into an analog signal;
a differential amplifier including
a first input terminal coupled to an output of the multiplexer system,
a second input terminal coupled to an output of the DAC, and
an output terminal;
an analog-to-digital converter (ADC) coupled to the output terminal of the differential amplifier; and
a digital signal processor (DSP) coupled to an output of the ADC, wherein the DSP is operative to extract an unwanted low frequency portion of signals from the amplifiers.

46. The micro system of claim 43, further comprising:
a plate; and
a plurality of actuators connected between the plate and the chip, the actuator operative to expand in response to receiving a signal.

47. The micro system of claim 46, wherein the actuator comprises a microbattery including a solid state electrolyte.

48. The micro system of claim 43, wherein the DSP is further operative to estimate a spectral structure the LFP activity.

49. The micro system of claim 48, wherein the DSP is further operative to generate feature vectors from the spectral structure of the LFP activity.

50. The micro system of claim 48, wherein the DSP is further operative to estimate a spectral structure of signals representative of single unit activity in the signals from the amplifiers.

51. The micro system of claim 50, wherein the DSP is further operative to generate feature vectors from the spectral structure of the LFP activity and the single unit activity.

52. A micro system adapted to be implanted subcutaneously on the skull of a subject, the micro system comprising:
a chip including a plurality of amplifiers arranged in an array;
a connector operative to couple each of a plurality of electrodes implanted in the subject's brain to a corresponding one of the amplifiers; and
a high pass filter operative to pass signals representative of local field potential (LFP) activity.

53. The micro system of claim 52, wherein the high pass filter comprises a look-up table including an offset value for each amplifier in the array.

54. The micro system of claim 53, further comprising:
a multiplexer system coupled to each amplifier in the array and operative to output a stream of data comprising signals sampled from amplifiers in the array;
a digital-to-analog converter (DAC) coupled to an output of the look-up table and operative to convert an offset value from the look-up table into an analog signal;
a differential amplifier including
a first input terminal coupled to an output of the multiplexer system,
a second input terminal coupled to an output of the DAC, and
an output terminal;
an analog-to-digital converter (ADC) coupled to the output terminal of the differential amplifier; and
a digital signal processor (DSP) coupled to an output of the ADC, wherein the DSP is operative to extract an unwanted low frequency portion of signals from the amplifiers.

55. The micro system of claim 52, further comprising an antenna operative to transmit signals from the micro system
